(19) United States
(12) Patent Application Publication
(10) Pub. No.: US 2016/0166658 A1
(43) Pub. Date: Jun. 16, 2016

(54) THERAPEUTIC INTERVENTION

(71) Applicant: ULC BUSINESS PLC, London, Greater London (GB)

(72) Inventors: Parashkev NACHEV, London (GB); Geraint REES, London (GB)

(21) Appl. No.: 14/907,737

(22) PCT Filed: Jul. 17, 2014
(86) PCT No.: PCT/GB2014/052193

§ 371 Date: Jan. 26, 2016

(30) Foreign Application Priority Data
Jul. 26, 2013 (GB) 1313356.6

(51) Int. Cl. A61K 38/48; A61N 1/36

(52) U.S. Cl. CPC A61K 38/4893; A61N 1/36096; C12Y 304/24069

(57) ABSTRACT

Provided are methods and materials for treating a neurological, behavioural, psychological, psychiatric, or personality disorder or syndrome in an individual, which disorder or syndrome is associated with a plurality of aberrant thoughts, behaviors and/or dispositions to behaviour. The methods comprise (i) selecting an individual suffering from, or believed to suffer from, said disorder or syndrome; and (ii) selecting a set of effector junctions in the individual, wherein at least one of said aberrant thoughts, behaviors or dispositions is modifiable by modulation of transmission across the set of effector junctions; (iii) treating the individual with an agent (for example botulinum toxin or a derivative or analog thereof) or intervention which causes blockade of or interference with the set of effector junctions, wherein blockade of the set of effector junctions results in an inhibition of the plurality of aberrant thoughts, behaviors and/or dispositions associated with the disorder.

### TECHNICAL FIELD

The present invention relates generally to methods and materials for use in modifying thought and behaviour, particularly aberrant thought and behaviour associated with or symptomatic of a syndrome or disorder, or itself leading to a syndrome or disorder

### BACKGROUND ART

Numerous behavioural disorders or syndromes have been characterised in the literature. Their manifestations include experiencing or expressing inappropriate or exaggerated types of thought, behaviour or emotion in relation to circumstances, or a general pervasive disposition to negative thoughts behaviour and emotion. They may also manifest by their physical consequences, such as obesity as a result of overeating, or self-harm as a result of emotional lability. Treatment of such disorders or syndromes is challenging. A non-limiting list of such disorders includes:

Emotional lability. A range of psychiatric and neurological disorders (including bipolar disorder, personality disorders, stroke, multiple sclerosis, traumatic brain injury, and motor neuron disease) affecting an estimated 6.4% of the population is associated with a behavioural disturbance characterised by a tendency to express emotion to a greater degree than circumstances compel, often rapidly changing from one intense expression to another. These patients cry at things they consider not to be sufficiently sad, or laugh at things that are not amusing, often within the same narrow timeframe. This emotional instability may be socially embarrassing or otherwise problematic in their daily lives. Conventional drug treatments are poorly effective.

Intermittent explosive disorder. An estimated 7.3% of general population will respond with extreme anger in circumstances that do not merit it, in ways that may be psychologically or physically damaging to themselves or to others. There is no effective drug treatment.

Agoraphobia and social phobia. At least 10% of the population is affected by psychological difficulties in circumstances involving actual or potential social relations with others, resulting in social withdrawal and consequently interfering with the ability to lead a productive and fulfilling life.

Overeating. Obesity—affecting ˜30% of people in the western world—is primarily a disorder of appetite not of weight. The key to obesity is therefore the reduction of appetite, for which there are no generally effective non-operative therapies available. Other overeating disorders include bulimia, for which again is not readily treatable.

Thus it can be seen that a novel approach to treating emotional and behavioural disorders or syndromes would provide a contribution to the art.

### DISCLOSURE OF THE INVENTION

The present invention is based on a novel approach to modifying such undesirable behaviours (and therefore their consequences) arising by exploiting the intrinsic synergy between dispositions and behaviours (see Example 1). The invention utilises effector junction (neuromuscular or neuroglandular) blockade to modify certain aspects of manifested behaviours (“leveraged behaviours”) which are constitutive of particular (target) dispositions. Blockade of an effector junction results in two things: first, the effector—the muscle or gland—either ceases to operate in response to the neural signal that normally drives it or operates to a lesser extent. Second, consequently on the first, the neural signal normally sent back to the brain to provide feedback that the effector is active is commensurately attenuated—because there is no or less effector action to feed back on. This feedback step has the potential to alter behaviour involving other effectors, for the brain's response in any circumstances is dependent on integration of feedback from all parts of the body potentially relevant to the response.

Thus blockade of the leveraged behaviour, modifies the disposition itself, thereby modifying a wider range of (target) behaviours involving effectors beyond the one that was subject to direct blockade.

In summary, the practice of the invention has three components: the target behaviours one wishes to inhibit, the target disposition giving rise to them, and the “leverage” behaviours through which one can inhibit the target disposition and thereby the target behaviours it gives rise to. The effect is produced by blocking the effector of the “leverage” behaviour.

As explained below, blockade can be achieved in a number of ways. However in a preferred embodiment it is achieved using a pharmacological junction blocker such as botulinum toxin or a derivative thereof.

Botulinum toxin is well known in the art for both therapeutic and cosmetic use. However the present utility is quite different from these earlier uses. For example the previous use of botulinum toxin and its analogues in cosmetic medicine and neurology has generally been aimed at attenuation of the action of the muscle itself, either for the sake of attenuating the movement it causes (e.g. in torticollis) or the incidental accompaniments of such attenuation (e.g. smoothing the wrinkles of the overlying skin).

It is also distinct from the use of botulinum toxin in the amelioration of anxiety and depression through blockade of frowning (see Wollmer et al. “Facing depression with botulinum toxin: A randomized controlled trial”, Journal of Psychiatric Research 46, 574-581 (2012); also U.S. Pat. No. 7,758,872). This prior art is concerned with the effect on low mood, as indexed by questionnaire-based diagnostic instruments, of blockade of the muscles involved in frowning. The present invention is not simply concerned with the alteration of mood but, but the alteration of a specific set of behaviours through the specific dispositions that lie behind them (see Example 6).

Thus in one aspect there is provided a method for treating a neurological, behavioural, psychological, psychiatric, or personality disorder or syndrome in an individual, which disorder or syndrome is associated with a plurality of aberrant thoughts, behaviors and/or dispositions to behaviour, the method comprising

(i) selecting an individual suffering from, or believed to suffer from, said disorder or syndrome; and

(ii) selecting a set of effector junctions in the individual, wherein at least one of said aberrant thoughts, behaviors or dispositions is (directly) modifiable by modulation of transmission across the set of effector junctions;

(iii) treating the individual with an agent or intervention which causes blockade of or interference with the set of effector junctions,
wherein blockade of the set of effector junctions results in an inhibition of the plurality of aberrant thoughts, behaviors and/or dispositions associated with the disorder, either directly or by modifying the disposition to the plurality of thoughts and behaviours.

Typically, as explained above, a subset or individual effector junction, controlling a subset or individual “leveraged” behaviour, is subject to direct intervention with an agent or other direct intervention described below. However the plurality of aberrant thoughts, behaviors, and dispositions modified by the present invention will be dependent on other effector junctions, which are not directly intervened with, but which are influenced owing to the change in the disposition to the behaviour as a result of altered feedback from the effectors that are blocked as explained above. This feedback may be embodied in the effector tissue itself (e.g. via proprioceptive spindles in muscle) or via sensors in other parts of the body which react to the operation of the leveraged behaviour.

### Behavioural Disorders or Syndromes

As set out in the Examples appended hereto, the invention may be applied to a variety of behavioural disorders, symptoms, syndromes, or diseases of interest. In each case selective effector junction blockade is utilised to modify the abnormal disposition lying at the root of the behaviour, thereby modifying a range of behaviours associated with that disposition. The blockade is applied directly to non-essential “leverage” effectors tightly associated with the target disposition and reinforcing of it through feedback which the intervention interferes with.

Preferred “leverage” behaviours addressable by the present invention are those which are involuntary or semi-involuntary in nature, i.e. either not under the control of will or only to a limited extent. There are two reasons for this: first, because such behaviours cannot easily be addressed psychologically—being involuntary—and second, because they are generally driven by evolutionarily primordial systems that have broad and powerful dispositional effects. The dispositions giving rise to them are thus believed to be particularly susceptible to intervention according to the methods of the present invention.

A non-limiting list of disorders to which the treatment can be applied includes emotional lability in the context of psychiatric, neurological, and personality disorders, intermittent explosive disorder, agoraphobia and social phobia, and overeating.

More specifically, a non-limiting list of such disorders include, with ICD10 codes in brackets:

Psychiatric disorders: agoraphobia (F40.0), social phobia (F40.1), bulimia nervosa (F50.2 and F50.3), emotionally unstable personality disorder (F60.3), histrionic personality disorder (F60.4), intermittent explosive disorder (F63.8).

Neurological/psychiatric disorders: dementia (especially F01 and F02), organic emotionally labile disorder (F06.6), and organic personality disorder (F07).

Neurological disorders: those thoset cause damage to the descending pathways from cortex to brainstem including encephalitis (G04), degenerative neurological disorders (G10, G12.2, G13), demyelination (G35, G36, G37), cerebrovascular disease (G46), and brain tumours (G71).

Behavioural disorders: obesity (E66).

### Effector Junctions

An “effector junction” is a junction between the final nerve in a neural pathway and the effector—muscle or gland—whose activity it governs. Junctions are thus embedded in the effector organs, within the tissue itself. Where the effector is a muscle the response is a muscular contraction, where it is a gland, the response is secretion from the gland. These junctions will generally—directly—effect the leveraged behaviour.

The present invention is preferably utilised with non-essential effect junctions which are required by, or result in, the leveraged behavious. By “non-essential” is meant that the inhibition of these junctions and behaviours does not cause harm or adverse physiological or pathological effect for the individual.

### Blockade of an Effector Junction

Blockade or blocking of effector function can be accomplished in several ways. Unless context demands otherwise the term “block” or “blocking” herein is used synonymously with “blockade”. None of these terms is used to denote or require “complete” blocking i.e. they include partial inhibition of function, provided the blockade is sufficient to modify the behaviour and thus disposition.

Preferably the effector junction is accessed directly by focal invasive means, through injection, targeted either anatomically or through functional or structural properties of the tissue determined with an appropriate device (see below).

In one embodiment blockade may be effected by electrical stimulation of the nerve via an implanted electronic device that modifies the excitability of the nerves feeding into the junction locally—e.g. using devices approaches such as that described by Ridding et al “Changes in muscle responses to stimulation of the motor cortex induced by peripheral nerve stimulation in human subjects” 2000, Experimental Brain Research pp 135-143.

A preferred agent for blockade is a substance that binds tightly and irreversibly to the junction where it is introduced, and which allows highly focal blockade.

Preferred agents interfere with release of a neurotransmitter at the junction e.g. acetylcholine. For example they may prevent release of the neurotransmitter e.g. by proteolysis of proteins involved in said release e.g. to preventing vesicles from releasing it. This can be achieved by botulinum toxin and its derivatives and analogues.

As is well known in the art botulinum toxin (BTX) is a toxin produced by the bacterium Clostridium botulinum, but which may either be obtained from a natural source or made by synthetic means.

The seven serologically distinct toxin types are designated A through G. Additionally, six of the seven toxin types have subtypes with five subtypes of toxin A having been described. The toxin is a two-chain polypeptide with a 100-kDa heavy chain joined by a disulfide bond to a 50-kDa light chain. This light chain is an enzyme (a protease) that attacks one of the fusion proteins (SNAP-25, syntaxin or synaptobrevin) at a neuromuscular junction, preventing vesicles from anchoring to the membrane to release acetylcholine. By inhibiting acetylcholine release, the toxin interferes with nerve impulses and causes flaccid (sagging) paralysis of muscles in botulism.

Toxin derivatives or analogs (or other agents of the present invention) will share identity with the native types, and also have the same biological activity i.e. proteolysis of relevant proteins at a neuromuscular junction, preventing vesicles from anchoring to the membrane to release acetylcholine. The term “analog” includes any polypeptide having an amino acid residue sequence substantially identical to the sequence specifically in which one or more residues have been substituted, preferably conservatively substituted with a functionally similar residue and which displays this same biological activity. Examples of conservative substitutions include the substitution of one non-polar (hydrophobic) residue such as isoleucine, valine, leucine or alanine for another, the substitution of one polar (hydrophilic) residue for another such as between arginine and lysine, between glutamine and asparagine, between threonine and serine, the substitution of one basic residue such as lysine, arginine or histidine for another, or the substitution of one acidic residue, such as aspartic acid or glutamic acid for another. “Derivatives” may be fragments or other modified BTX sequences which again retain the biological activity. Typically the agents of the present invention will share at least 50%, at least 75%, at least 85%, at least 90%, at least 95%, at least 96%, at least 97%, at least 98%, or at least 99% identity with the amino acid sequences of the native BTX agents. Percentage identity can be assessed using ClustalW2 at the European Bioinformatics Institute website (part of EMBL) using fast pairwise default alignment options (KTUP 1; Window length 5; Score Type %; Topdiag 5; Pairgap 3).

As set out in U.S. Pat. No. 7,758,872, BTX reversibly paralyzes striated muscle when administered in sub-lethal doses. BTX has been used in the treatment in a number of neuromuscular disorders and conditions involving muscular spasm including, but not limited to, dystonia, hemifacial spasm, tremor, spasticity (e.g. resulting from multiple sclerosis), anal fissures and various ophthalmologic conditions (for example, see Carruthers et al., J. Amer. Acad. Derm. 34:788-797, 1996).

BTX is a generic term covering the family of toxins produced by C. botulinum comprising, and in particular the eight serologically distinct forms A, B, C1, C2, D, E, F and G. These toxins are among the most powerful neuroparalytic agents known (c.f. Melling et al., Eye 2:16-23, 1988). Serotypes A, B and F are the most potent (Boyer et al., “Botulinum Toxin Type B: Experimental and Clinical Experience,” in Therapy With Botulinum Toxin, Marcel Dekker, Inc Jankovic et al. (eds.), pgs. 71-85, 1994).

Botulinum toxin type A can be obtained by establishing and growing cultures of Clostridium botulinum in a fermenter and then harvesting and purifying the fermented mixture in accordance with known procedures. Alternatively, the Botulinum toxin serotypes are initially synthesized as inactive single chain proteins which must be cleaved or nicked by proteases to become neuroactive. High quality crystalline Botulinum toxin type A can be produced from the Hall A strain of Clostridium botulinum. The Schantz process can be used to obtain crystalline Botulinum toxin type A (see Schantz et al., Microbiol Rev. 56; 80-99, 1992). Generally, the Botulinum toxin type A complex can be isolated and purified from an anaerobic fermentation by cultivating Clostridium botulinum type A in a suitable medium. This process can be used, upon separation out of the non-toxin proteins, to obtain pure Botulinum toxins, such as for example: purified Botulinum toxin type A with an approximately 150 kD molecular weight, purified Botulinum toxin type B with an approximately 156 kD molecular weight and purified Botulinum toxin type F with an approximately 155 kD molecular weight.

Botulinum toxins and/or Botulinum toxin complexes can be obtained from List Biological Laboratories, Inc., Campbell, Calif.; the Centre for Applied Microbiology and Research, Porton Down, U.K.; Wako, Osaka, Japan; Metabiologics, Madison, Wis.) as well as from Sigma Chemicals, St Louis, Mo.

A Botulinum toxin type A complex has been approved by the U.S. Food and Drug Administration for the treatment of blepharospasm, strabismus and hemifacial spasm.

BOTOX™ (Botulinum toxin A, Allergan, Inc., Irvine, Calif., U.S.A.) is sold in 100 Unit vials. DYSPORT™ (Speywood Pharmaceuticals, Ltd., Maidenhead, U.K.) is sold in 500 Unit vials. For cosmetic uses, the vial contents are typically diluted with 1 or 2 ml of sterile saline solution, which for BOTOX™ provides a 100 or 50 Unit/ml dilution. DYSPORT™ BTX-A is roughly tenfold less toxic than BOTOX™ and approximately fourfold greater amounts of the DYSPORT™ product will usually be injected to achieve the same result as would be obtained using a specific number of Units of BOTOX™.

### Identification of Leverage Effector

The practice of the present invention requires that the appropriate effector junction be identified.

The effector junction may be a neuromuscular junction or a neuroglandular one. In the light of the present disclosure those skilled in the art will be able to identify the effector associated with the relevant behaviour, and therefore disposition.

In a preferred embodiment the methods of the present invention comprise a first step of identifying a target tissue in which the target effector junction is present; preferably this identification utilises a device e.g. one which is measures the electrophysiology of the tissue (e.g. as electromyography; Kamen, Gary. Electromyographic Kinesiology. In Robertson, DGE et al. Research Methods in Biomechanics. Champaign, IL: Human Kinetics Publ., 2004, or uses functional or structural imaging (e.g. ultrasonography; see e.g. Ultrasound Guided Needle Insertion and Injection Skills—Ki Jinn Chin—ISURA 2012 as www.usra.ca website).

For example the effector can be identified anatomically, based on its location, and/or neurophysiologically, based on its functional properties as assessed by surface or depth electrodes, or other laboratory tools sensitive to effector function. If required, electromyographic (EMG) guided needles may be used for injection to determine needle location of a high degree of accuracy.

Many electrode devices are known in the art for electrophysiology mapping—see e.g. US2009/0163801. In a typical system, a catheter may be inserted within a vessel located near the surface of a body (e.g., in an artery or vein in the leg, neck, or arm) and manoeuvred to a region of interest within the body. An electrode disposed at one end of the catheter detects changes in electrical potential resulting from the transmission of electrical signals between points on the body. Signals generated by the electrode are then used to generate an image of a tissue surface.

Preferably the device is one which measures muscle tone (e.g. facial muscle tone) using electrophysiology.

Preferably the device is a portable one. Preferably it is a portable ambulatory device which can be worn over a period of time so as to capture its ecological variation in facial muscle tone. This not only permits identification of the e.g. muscles (and hence junctions) to be targeted, but also an assessment of the required dose of blockade agent or intervention to be assessed. Such devices are commercially available—see e.g. “Validation of a portable EMG device to assess muscle activity during free-living situations” T. J. Walters, K. A. Kaschinske, S. J. Strath, A. M. Swartz, K. G. Keenan, Journal of electromyography and kinesiology: official journal of the International Society of Electrophysiological Kinesiology 8 Jul. 2013.

### Regimes & Dosage Forms

For applications of BTX, total dose per treatment can be varied and depends upon the condition being treated and the site of application of BTX. For example, a typical total dose of 10-50 Units (such as about 20 to about 40 Unit equivalents) may be used.

A “Unit equivalent” is an amount of BTX that is equivalent to standard Units of Botulinum Toxin A. A standard Unit of BTX-A is defined as the mean LD50 for female Swiss Webster mice weighing 18-20 grams (Schantz and Kaultner, J. Assac. Anal. Chem. 61: 96-99, 1978). The estimated human LD50 for a 70-kg persan is 40 Units/kg or about 2500-3000 Units.

An exemplary injection technique involves the use of a short, narrow needle (e.g. [½] inch or 8 mm; 30-gauge) with an insulin- or tuberculin-type syringe. Subjects are typically treated in the seated position. The skin area is cleaned with an alcohol swab. A single syringe may be used for multiple injections to treat different locations in a single muscle or different locations on a patient's face. Typically, the plunger of the syringe is depressed as the needle is withdrawn so that toxin is evenly distributed at the injection site. Pressure or gentle massage may be applied at the injection site to assist in dissipating the toxin. The toxin will typically migrate approximately 1 cm from the site of injection.

Onset of muscle paralysis following injection usually occurs within hours of treatment. The duration of paralysis will vary from patient to patient. Typically, duration will be from two to eight months, for example about three to about six months, or for example about three months, before subsequent treatment is required to modify the behavior, although Botulinum toxin type A can have an efficacy for up to 12 months (Naumann et al., European J. Neurology 6(Supp 4):S111-S115, 1999).

Generally speaking the treatments of the present invention will be long-term treatment, repeated as necessary when the effect of the drug wears out. The duration of treatment will depend on each individual case, to be guided clinically.

Any sub-titles herein are included for convenience only, and are not to be construed as limiting the disclosure in any way.

The invention will now be further described with reference to the following non-limiting Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### EXAMPLES

### Example 1

### Principle of the Invention

Human behaviour is conventionally understood as the manifestation of neural processes that are themselves independent of the behaviour, in the same way as a driver is independent of the vehicle he is driving. This is mistaken. In reality, the disposition to behave one way or another is inextricably bound up with the manifestation of the behaviour itself. For example, when one cries, the tears are not a manifestation of sadness, but constitutive of sadness. Though seemingly trivial, this distinction is of crucial importance. Whereas dispositions are poorly accessible, being dependent on neural processes largely opaque to analysis and manipulation, behaviour, being on the surface, can be modified directly.

If the behaviour is constitutive of the disposition, reinforcing it by feedback, then blocking all or even part of the behaviour may block the disposition itself, thereby blocking other manifestations of the disposition. Thus blocking the capacity to generate tears may block the sadness that seemingly only drives them but is in fact also itself driven by them. We can exploit this fundamental reciprocity between dispositions and behaviour to modify dispositions by modifying a manipulable aspect of the behaviour, a “leverage” behaviour that is associated with them. Crucially, we need only block a limited aspect of a behaviour to have an effect on the general disposition of which it is an instance as long as the association between the two is strong. Returning to the example of sadness, though crying is only a limited aspect of the behavioural repertoire of sadness, it is very strongly associated with it. An easily practicable and highly focal intervention such as blocking tear generation can thus have a general effect on a broad disposition to a much greater range of behaviour.

The strength of association on the grounds of which candidate targets for blockade may be identified is empirically determinable; such targets are also, however, predictable from the nature of the dispositions and associated behaviours. Broad dispositions of clinical and psychological interest are generally driven by evolutionarily primordial systems such as those we identify with basic instincts and emotions. Such fundamental dispositions are almost always strongly associated with characteristic involuntary or semi-voluntary physical behaviours that are readily amenable to manipulation, e.g. sadness with crying, anger with snarling, anxiety with hunching one's shoulders, and so on. Blocking these largely automatic behaviours can thus alter the target disposition without interfering with the voluntary behaviour—the fundamental life of the patient—that the invention seeks to improve.

More specifically, blockade of an effector junction results in two things: first, the effector—the muscle or gland—either ceases to operate in response to the neural signal that normally drives it or operates to a lesser extent. Second, consequently on the first, the neural signal normally sent back to the brain to provide feedback that the effector is active is commensurately attenuated—because there is no or less effector action to feed back on. Such feedback may be embodied in the effector tissue itself (for example via proprioceptive spindles in muscle) or via sensors in other parts of the body (for example via sensation of liquid in the mouth in the case of salivary gland function). This feedback step has the potential to alter behaviour involving other effectors, for the brain's response in any circumstances is dependent on integration of feedback from all parts of the body potentially relevant to the response.

In the non-limiting Examples below we identify a set of target automatic physical behaviours—some motor, others glandular—whose blockade with focal effector junction blockers such as botulinum toxin may attenuate a corresponding disposition of interest.

### Example 2

### Emotional Lability

Emotional lability is characterised by an exaggeration of normal emotional responses to an extent that is distressing to the patient or those around him. It may accompany a wide range of neurological disorders that affect the central nervous system as previously outlined, and is a feature of both occurrent and dispositional psychiatric and psychological disorders, especially cluster B personality disorders. Since it is an exaggeration of many rather than just one emotion, the application of the method here would be to attenuate (rather than completely block) the facial musculature recruited by facial expression generally, where appropriate targeting only the muscles associated with the set of expressions that are most problematic. Possible target muscles may include but are not limited to frontalis, procerus, zygmaticus, levator labii superiori, levator anguli oris, dilator naris and depressor septi, corrugator, and platysma. Attenuation of the emotional lability can thus be achieved without complete paralysis of the facial musculature, thus allowing normal facial expression to be maintained. The dose of blocking agent used would depend on the nature of the agent. Note, the treatment does not merely mask the expression, but modifies the disposition to emote excessively.

### Example 3

### Intermittent Explosive Disorder

People with intermittent explosive disorder are prone to outbursts of anger sometimes associated with violence. When considering the control of verbal violence in the context of intermittent explosive disorder the following strategy is adopted:

The target behaviour is the verbal output, consisting of utterances of certain tone and content. To inhibit this behaviour directly one would either have to paralyse the muscles involved in speech or else inhibit the thoughts that lead to it. The former obviously cannot be made specific to the target behaviour, the latter requires pharmacological or cognitive-behavioural therapy which is the current—and largely ineffective—approach.

However, one can inhibit the target behaviour by inhibiting other behaviours strongly associated with it, such as the characteristic facial expression that accompanies aggression. These “leverage” behaviours may take the form of any physical response: muscular contraction in facial expression or in posture, glandular secretion in salivation or perspiration, etc. What characterises them is that they are tightly associated with the disposition giving rise to the target behaviours, usually but not always in an involuntary or semi-involuntary manner. Blockade of such “leverage” behaviour successfully inhibits the target behaviour because it removes part of the feedback to the brain that normally reinforces the disposition to the target behaviour, thereby weakening its drive. Crucially, blockade of these “leverage” behaviour effectors such as the facial muscles associated with the expression of aggression need not have any deleterious effects as they need not be critically involved in any important function. For example, the muscles associated with the facial expression of anger have little use in expressions of positive emotions. Thus, one can generally inhibit a target behaviour indirectly by blocking non-essential, leverage behaviours tightly associated with it, exploiting the break of feedback reinforcement of behaviour from such other, non-essential effectors.

More specifically, anger has a highly characteristic facial expression involving contraction of levator labii superioris and nasalis. Thus blocking or antagonising the general disposition to anger can be effected by blocking or attenuating the action of these and other muscles specifically associated with anger.

### Example 4

### Agoraphobia and Social Phobia

What is common to these disorders is a resistance to engaging in activities either involving social contact or the potentiality of social contact. Such resistance is commonly associated with a typical bodily posture characterised by raising of the shoulders, mostly through the action of the upper part of the trapezius muscle bilaterally. When considering these behaviours, the synergy between the disposition and the behaviour is reinforced by the fact that the underlying disposition is modified by the opinion of others resulting from the behaviour, observed from outside. This tends to increase the degree to which the behaviour is manifest.

For example posture, and in particular the position of the shoulders, not only reflects the disposition, but also affects the manner in which the sufferer is treated by others. Hunching one's shoulders in a defensive posture is constitutive of agoraphobia and social phobia, and so blocking the movement ameliorates the abnormality of disposition it indicates. In the practice of the invention, the upper segments of the trapezius muscles bilaterally that elevate the shoulder are blocked. This is functionally of little consequence to the operation of the shoulder itself, elevation of the shoulder generally being if anything counterproductive to effective movement.

### Example 5

### Overeating

Obesity is most commonly caused by increased appetite: in short, overeating. Eating involves a number of constitutive behaviours that reinforce its practice: chief amongst those is is salivation. The sight and/or thought of food is associated with salivation, which in turn reinforces the desire for food. Attenuating the salivation reflex can thus reduce the desire for food.

The salivation reflex is attenuated by reducing saliva production from the salivary glands using a neuroglandular blocker such as botulinum toxin. Attenuating the salivation reflex e.g. by targeting the parotid gland, attenuates the underlying disposition (desire for food) and can therefore lead to sustained weight loss. The subject is one who suffers any disorder of overeating (e.g. obesity or bulimia) and may or may not be one diagnosed as being obese.

Botox has previously been used to treat hypersalivation (see Ellies et al “Botulinum toxin to reduce saliva flow: selected indications for ultrasound-guided toxin application into salivary glands. Laryngoscope. 2002 January; 112(1):82-6” but that publication was not concerned with treating overeating disorders. Indeed all previous prior art in relation to glandular blockade has been for the reduction of pathologically excess salivation for its own sake, not the modulation of normal salivation with the aim of reducing an abnormally enhanced appetite.

### Example 6

### Discussion of Prior Art Use of Botulinum Toxin in the Amelioration of Anxiety and Depression Through Blockade of Frowning

This earlier work (see Wollmer et al. “Facing depression with botulinum toxin: A randomized controlled trial”, Journal of Psychiatric Research 46, 574-581 (2012); also U.S. Pat. No. 7,758,872) was concerned with the effect on ‘low mood’, as indexed by questionnaire-based diagnostic instruments, of blockade of the muscles involved in frowning.

This is conceptually distinct from the present invention, which is not concerned with the alteration of mood per se but, but the alteration of a specific set of behaviours through the specific dispositions that lie behind them.

In particular, a disposition to behave in a particular way is not reducible to any set of moods, though a mood may occasionally be cited in the explanation of a behaviour. For example, there is no such thing as an emotionally labile mood, for lability is a disposition to exhibit any emotion to excess in circumstances that provoke it. Someone with emotional lability need not have an abnormal mood at any point, and a change to a low mood or to a high mood can not be used to ‘explain’ lability, which is a propensity to express emotion to excess whatever its valence, independently of current mood. Emotion itself is not a mood—an intransitive state—but simply an emotion—a transitive state with a specific object (one cries at something, laughs at something, etc)—and so altering the expression of an emotion is not altering a mood.

Equally, there is no such thing as an intermittently explosive mood, for intermittently explosive outbursts occur in the context of normal mood inbetween outbursts, and the outburst itself is an emotion or a behaviour—anger, contempt, aggression, violence—not a mood. Similarly, agoraphobia and social phobia are not moods but dispositions to avoidance in the context of specific situations involving or likely to involve social contact. Neither the behaviour—avoidance—nor the disposition are charactersable as moods. Overeating cannot be described as a mood or an emotion of any kind, and the modulation of overeating described in the Examples herein does not involve as the effective step a change in mood of any kind.

1. A method for treating a neurological, behavioural, psychological, psychiatric, or personality disorder or syndrome in an individual, which disorder or syndrome is associated with a plurality of aberrant thoughts, behaviors and/or dispositions to behaviour, the method comprising
(i) selecting an individual suffering from, or believed to suffer from, said disorder or syndrome; and
(ii) selecting a set of effector junctions in the individual, wherein at least one of said aberrant thoughts, behaviors or dispositions is modifiable by modulation of transmission across the set of effector junctions;
(iii) treating the individual with an agent or intervention which causes blockade of or interference with the set of effector junctions,
wherein blockade of the set of effector junctions results in an inhibition of the plurality of aberrant thoughts, behaviors and/or dispositions associated with the disorder.

2. A method as claimed in claim 1 wherein the behaviours are involuntary or semi-involuntary in nature.

3. A method as claimed in claim 1 wherein the effector is a neuromuscular junction.

4. A method as claimed in claim 1 wherein the effector is a neuroglandular junction.

5. A method as claimed in claim 1 wherein the disorder is an eating disorder leading to an abnormally enhanced appetite and excessive eating, and the effector junction is one which activates saliva production in the salivary glands.

6. A method as claimed in claim 5 wherein the disorder is bulimia or one which leads to clinical obesity.

7. A method as claimed in claim 1 wherein the disorder is emotional lability leading to an exaggeration of emotional responses, and the effector junction is one which activates facial musculature whose contraction is characteristic of at least one of the emotional responses.

8. A method as claimed in claim 1 wherein the disorder is intermittent explosive disorder leading to expression of anger, hatred or extreme contempt, and the effector junction is one which activates the facial musculature whose contraction is characteristic of anger.

9. A method as claimed in claim 1 wherein the disorder is a disorder of anxiety and/or social withdrawal, and the effector junction is one which is present in the shoulder, neck and head musculature whose contraction is characteristic of the posture associated with such disorder.

10. A method as claimed in claim 1 wherein the method comprises further administering to the individual a therapeutically effective amount of an additional modality of treatment for the disorder.

11. A method as claimed in claim 1 wherein the treatment is with an agent which causes blockade of the effector junction.

12. A method as claimed in claim 11 wherein the agent is pharmacological agent causing long term interruption of junction function through chemical interference with the junction.

13. A method as claimed in claim 11 wherein the agent interferes with release of a neurotransmitter.

14. A method as claimed it claim 13 wherein the agent is botulinum toxin or a derivative or analog thereof.

15. A method as claimed in claim 14 wherein the botulinum toxic is serotype A, B or F.

16. A method as claimed in claim 15 wherein about 20-40 Units of Botulinum toxin type A is administered to the region of the effector junction.

17. A method as claimed in claim 1 wherein the treatment is with an electrical intervention causing long term interruption of effector junction function through electrical interference with the junction.

18. A method as claimed in claim 17 wherein the electrical intervention is via an implanted electronic device that modifies the excitability of the nerves feeding into the junction locally.

19. A method as claimed in claim 1 wherein the effector junction is targeted within a target tissue in step (ii) using electrophysiology or functional imaging.

20. A method as claimed in claim 1 wherein the effector junction is targeted within a target tissue in step (ii) using ultrasonography or any other structural imaging modality.

21. A method as claimed in claim 19 wherein step (ii) is carried out with device which measures muscle tone using electrophysiology.

22. A method as claimed in claim 21 wherein the device is a portable ambulatory device which can be worn by the individual over a period of time so as to capture its ecological variation in muscle tone.

23. A method as claimed in claim 22 wherein the muscle tone is facial muscle tone.

24. (canceled)

25. (canceled)

26. A device adapted for use in a method as claimed in claim 23.
